(19) United States
(12) Patent Application Publication
(10) Pub. No.: US 2025/0134951 A1
(43) Pub. Date: May 1, 2025

(54) OCTREOTIDE ACETATE SUSTAINED-RELEASE MICROSPHERE AS WELL AS PREPARATION METHOD THEREFOR AND USE THEREOF

(71) Applicant: SHANGHAI RUINING BIOTECHNOLOGY CO., LTD., Shanghai (CN)

(72) Inventors: Haiying XIA, Shanghai (CN); Bingyan WANG, Shanghai (CN); Zhanpeng ZHANG, Shanghai (CN); Zhen PAN, Shanghai (CN)

(21) Appl. No.: 19/007,609

(22) Filed: Jan. 2, 2025

Related U.S. Application Data
(63) Continuation of application No. PCT/CN2024/077044, filed on Feb. 8, 2024.

(30) Foreign Application Priority Data
Jul. 28, 2023 (CN) 202310947993.2

(51) Int. Cl. A61K 38/12; A61K 9/50

(52) U.S. Cl. CPC A61K 38/12; A61K 9/5031

(57) ABSTRACT

The present disclosure relates to the technical field of microsphere drug preparations, in particular to an octreotide acetate sustained-release microsphere as well as a preparation method therefor and use thereof. The preparation method includes the following steps: S1, dissolving a polylactic acid-co-glycolic acid copolymer in a first organic solvent to prepare an oil phase 1; and dissolving octreotide acetate in a second organic solvent to prepare an oil phase 2; S2, adding the oil phase 2 into the oil phase 1 under stirring conditions to obtain a suspension containing octreotide acetate; S3, transferring the suspension to an aqueous phase, and performing emulsifying to obtain a semi-cured drug-loaded microsphere; and S4, transferring the semi-cured drug-loaded microsphere to a curing phase, and removing the solvents to obtain the octreotide acetate sustained-release microsphere.

### CROSS-REFERENCE TO RELATED APPLICATIONS

The present application is a continuation of International Application No. PCT/CN2024/077044, with an international filing date of Feb. 8, 2024, which is based upon and claims priority to Chinese Patent Application No. 2023109479932, filed on Jul. 28, 2023, the entire contents of all of which are incorporated herein by reference.

### TECHNICAL FIELD

The present disclosure relates to the technical field of microsphere drug preparations, in particular to an octreotide acetate sustained-release microsphere as well as a preparation method therefor and use thereof.

### BACKGROUND

Octreotide is an artificially synthesized octapeptide derivative of natural somatostatin, which was first synthesized in 1979. The octreotide retains same pharmacological effects as growth hormone and is capable of inhibiting the secretion of many hormones, including secretin, cholecystokinin, glucagon, growth hormone (GH), insulin, pancreatic polypeptide, thyroid stimulating hormone, and vasoactive intestinal peptide, etc. Clinically, the octreotide is mainly used for treating acromegaly, gastrointestinal pancreatic endocrine tumors, etc., and has been widely used in clinical applications.

Two kinds of octreotide acetate preparations have been approved for marketing by the FDA. One is an injection that is administered by subcutaneous injection for 3 times a day; The other one is a reservoir type microsphere for injection that is administered by intramuscular injection for 1 time a month and has a longest administration period of 1 month. The 1-month preparation solves the problem of partial compliance of the single-day injection. However, the 1-month sustained-release octreotide acetate microsphere on the market needs to use a special glucose-PLGA star-shaped polymer (GLU-PLGA) as a matrix.

PLGA, a high polymer material, is commonly used as a carrier by domestic and foreign researchers, and a multiple emulsion method (W/O/W method) is usually selected to prepare octreotide acetate sustained-release microspheres. The octreotide acetate sustained-release microspheres have a series of problems in research and development, among which uneven particle sizes of the microspheres, a low encapsulation rate and serious burst release are main problems. The octreotide acetate sustained-release microspheres have some disadvantages in applications: as the particle size range of the microspheres is generally 1-500 um, the small particle size can be a few nanometers and the large particle size can reach 800 um, needles of injections required are relatively thick, and stimulations, such as pain, hard nodes or redness, will be caused at injection sites; as the particle sizes of the microspheres are uneven, drug release is affected; and when materials have poor degradability, inflammations are likely to be caused.

Some methods have been disclosed in patents to improve the sphericity and encapsulation rate of the microspheres, but have little effects. Chinese Patent CN102940609A discloses an octreotide sustained-release microsphere with a high encapsulation rate and a preparation method therefor. The encapsulation rate of the microsphere prepared is 46%. The microsphere has a large span in particle size and is uneven. Moreover, many auxiliary materials are added during preparation of the microsphere, and a process is complex.

Chinese Patent CN114948881A disclosed an octreotide acetate microsphere and a preparation method therefor. The octreotide microsphere is prepared by single emulsification, and the microsphere prepared has a relatively low encapsulation rate. In order to prepare microspheres with a high drug loading rate, a large amount of octreotide are required to be put into use, thereby increasing the input and cost of the drugs.

### SUMMARY

Aiming at the disadvantages of the prior art, a first aspect of the present disclosure provides a preparation method for an octreotide acetate sustained-release microsphere. The preparation method includes the following steps:
S1, dissolving a polylactic acid-co-glycolic acid copolymer (PLGA) in a first organic solvent to prepare an oil phase 1; and dissolving octreotide acetate in a second organic solvent to prepare an oil phase 2;
S2, adding the oil phase 2 into the oil phase 1 under stirring conditions to obtain a suspension containing octreotide acetate;
S3, transferring the suspension to an aqueous phase, and performing emulsification to obtain a semi-cured drug-loaded microsphere; and
S4, transferring the semi-cured drug-loaded microsphere to a curing phase, and removing the solvents to obtain the octreotide acetate sustained-release microsphere.

In some embodiments, a relative molecular mass of the polylactic acid-co-glycolic acid copolymer is 12,000-36,000, a molar ratio of lactic acid to glycolic acid is (50-75):(25-50), and an intrinsic viscosity is 0.15-0.45 dL/g.

Further, the relative molecular mass of the polylactic acid-co-glycolic acid copolymer is 24,000-36,000, the molar ratio of the lactic acid to the glycolic acid is 50:50, the intrinsic viscosity is 0.3-0.4 dL/g, and a preferred source is Shenzhen Lvbao Technology Co., Ltd.

The applicant has found in an experiment that parameters, especially the viscosity, of polylactic acid-co-glycolic acid have a significant impact on the morphology and particle size of the microsphere, too high or too low viscosity will not only result in a non-sphericity microsphere, but also may result in an uneven particle size. Only when the intrinsic viscosity is 0.3-0.4 dL/g, the microsphere has relatively good morphology and even particle size, long term stability and a highest encapsulation rate.

In some embodiments, the first organic solvent includes at least one of ethyl acetate, ethyl formate, methyl formate, methyl acetate, butanone, tetrahydrofuran, acetone, acetonitrile, dimethyl sulfoxide, dichloromethane, chloroform, trichloroethylene, ethylene glycol ether, and triethanolamine.

In some embodiments, the second organic solvent includes at least one of dimethyl sulfoxide, N,N-dimethylformamide, and N-methylpyrrolidone.

In some embodiments, both the aqueous phase and the curing phase are a buffer solution containing a surfactant and a metal salt.

Further, the buffer solution includes at least one of an acetic acid buffer, a phosphate buffer, and a citric acid buffer.

Further, a pH value of the buffer solution is 3.0-5.5, and furthermore, the pH value of the buffer solution is 4.5.

The pH value of the buffer solution has a significant impact on the encapsulation rate of the microsphere. The too high or too low pH value will cause a decrease in encapsulation rate. Only when the pH value is 4.5, the encapsulation rate is highest, and the reason may be that octreotide acetate has highest drug stability at this pH value.

In some embodiments, a mass percentage of polylactic acid-co-glycolic acid in the oil phase 1 is 30%-60%.

In some embodiments, the surfactant includes at least one of Tween, Span, PVA, poloxamer, and betaine.

Further, a mass by volume percentage concentration of the surfactant is 1%-10%, and preferably, the mass by volume percentage concentration of the surfactant is 3%.

Further, the metal salt includes, but is not limited to, common types in the art, such as at least one of sodium chloride or potassium chloride. A mass by volume percentage concentration of the metal salt is 5%-20%, and preferably, the mass by volume percentage concentration of the metal salt is 10%.

Further, S4 further includes performing centrifugation or filtration on the octreotide acetate sustained-release microsphere to remove the excess solvents and surfactant, performing washing with deionized water, performing repeated centrifugation and washing for 5 times or above, and performing freeze drying on the microsphere through a freeze dryer to finally obtain an octreotide acetate sustained-release microsphere freeze-dried powder.

A second aspect of the present disclosure provides an octreotide acetate sustained-release microsphere, wherein an encapsulation rate of the sustained-release microsphere is greater than 90%.

A third aspect of the present disclosure provides application of an octreotide acetate sustained-release microsphere in preparation of a drug.

A fourth aspect of the present disclosure provides a drug, wherein raw materials for preparation include an octreotide acetate sustained-release microsphere and a pharmaceutically acceptable excipient.

Compared with the prior art, the present disclosure has the following beneficial effects.
1. The octreotide acetate sustained-release microsphere with a high drug loading rate and a high encapsulation rate is prepared in the present disclosure. Experimental tests show that the encapsulation rate can reach 90% or above, the drug stability is good, and the bioavailability is high, such that octreotide acetate shows a higher application value and a more remarkable use effect.
2. The microsphere prepared in the present disclosure achieves stable sustained release in vitro, and a cumulative dissolution rate test shows that the release period of a water-soluble drug is prolonged.
3. The optimal pH value in the system is explored in the present disclosure, and the microsphere prepared has spherical and smooth surface morphology and a high encapsulation rate at the pH value of 4.5.
4. The optimal viscosity of the polylactic acid-co-glycolic acid copolymer in the system is explored in the present disclosure, and the microsphere prepared has best morphology, best stability and a highest encapsulation rate when the intrinsic viscosity is 0.3-0.4 dL/g.
5. The microsphere prepared in the present disclosure has uniform and narrow particle size distribution, raw materials for preparation are simple and easy to obtain, a preparation process is easy to scale-up, and a broad industrialization prospect is achieved.

### BRIEF DESCRIPTION OF THE DRAWINGS

FIG. 1 is an appearance morphology photo of a sustained-release microsphere in Example 1 of the present disclosure.

FIG. 2 is an appearance morphology photo of a sustained-release microsphere in Comparative Example 2 of the present disclosure.

FIG. 3 is a comparative diagram of the cumulative dissolution rate in Example 1, Comparative Example 1 and Comparative Example 2 of the present disclosure.

### DESCRIPTION OF THE EMBODIMENTS

### Example 1

A preparation method for an octreotide acetate sustained-release microsphere is provided. The preparation method includes the following steps:
S1, dissolving 2.3 g of a polylactic acid-co-glycolic acid copolymer in 5.5 g of ethyl acetate as a first organic solvent to prepare an oil phase 1; and dissolving 0.2 g of octreotide acetate in 0.8 g of dimethyl sulfoxide as a second organic solvent to prepare an oil phase 2; wherein a relative molecular mass of the polylactic acid-co-glycolic acid copolymer was 24,000-36,000, a molar ratio of lactic acid to glycolic acid was 50:50, an intrinsic viscosity was 0.3-0.4 dL/g, and a source was the same as the preferred source in the summary;
S2, adding the oil phase 2 into the oil phase 1 (mass ratio:oil phase 1:oil phase 2=7:1) under stirring conditions at a stirring speed of 500 rpm, and subjecting an obtained primary suspension to continuous stirring at 5,000 rpm for 5 minutes to obtain a suspension containing octreotide acetate;
S3, transferring the suspension obtained in S2 to 50 mL of an aqueous phase, and performing emulsifying to obtain a semi-cured drug-loaded microsphere; wherein the aqueous phase was an acetic acid buffer solution (pH value: 4.5) containing 3 wt % of a poloxamer surfactant and 10 wt % of sodium chloride; and
S4, transferring the semi-cured drug-loaded microsphere solution obtained in S3 to 100 mL of a curing phase, performing heating to 40° C., and maintaining a vacuum degree at 400 mbarr for 120 minutes; maintaining the temperature at 40° C., and increasing the vacuum degree to 150 mbarr continuously for 90 minutes to remove the solvents so as to obtain the octreotide acetate sustained-release microsphere, wherein the curing phase was an acetic acid buffer solution (pH value: 4.5) containing 16 wt % of a poloxamer surfactant and 10 wt % of a sodium chloride metal salt; and subjecting the octreotide acetate sustained-release microsphere to washing with deionized water, repeated centrifugation and washing for 5 times, and freeze-drying through a freeze dryer to finally obtain a sustained-release microsphere freeze-dried powder.

### Example 2

A preparation method for an octreotide acetate sustained-release microsphere is provided. A specific embodiment is the same as Example 1, but has the difference that the content of the polylactic acid-co-glycolic acid copolymer was 1.65 g.

### Example 3

A preparation method for an octreotide acetate sustained-release microsphere is provided. A specific embodiment is the same as Example 1, but has the difference that the content of the polylactic acid-co-glycolic acid copolymer was 3.3 g.

### Example 4

A preparation method for an octreotide acetate sustained-release microsphere is provided. A specific embodiment is the same as Example 2, but has the difference that the pH value of the acetic acid buffer solution was 3.0.

### Example 5

A preparation method for an octreotide acetate sustained-release microsphere is provided. A specific embodiment is the same as Example 2, but has the difference that the pH value of the acetic acid buffer solution was 5.5.

### Example 6

A preparation method for an octreotide acetate sustained-release microsphere is provided. A specific embodiment is the same as Example 1, but has the differences that the aqueous phase was an acetic acid buffer solution (pH value: 4.5) containing 1 wt % of a poloxamer surfactant and 15 wt % of sodium chloride; and the curing phase was an acetic acid buffer solution (pH value: 4.5) containing 10 wt % of a poloxamer surfactant and 20 wt % of a sodium chloride metal salt.

### Comparative Example 1

A preparation method for an octreotide acetate sustained-release microsphere is provided. A specific embodiment is the same as Example 1, but has the differences that the preparation method includes the following steps:
S1, weighing and dissolving 3 g of a polylactic acid-co-glycolic acid copolymer in 7 g of ethyl acetate to prepare an oil phase; and weighing and dissolving 0.25 g of octreotide acetate in 1 g of ultrapure water to prepare an internal aqueous phase;
S2, slowly adding the internal aqueous phase dropwise into the oil phase (mass ratio: internal aqueous phase:oil phase=7:1) at a stirring speed of 500 rpm, and subjecting an obtained primary suspension to continuous stirring at 5,000 rpm for 5 minutes to form a suspension containing drug particles; and
S3, transferring the suspension obtained in S2 to 50 mL of an external aqueous phase containing 3 wt % of a poloxamer surfactant, performing emulsifying and curing, performing heating to 40° C., and maintaining a vacuum degree at 400 mbarr for 120 minutes; maintaining the temperature at 40° C., and increasing the vacuum degree to 150 mbarr continuously for 90 minutes to remove the solvent so as to obtain the octreotide acetate sustained-release microsphere; and subjecting the octreotide acetate sustained-release microsphere to washing with deionized water, repeated centrifugation and washing for 5 times, and freeze-drying through a freeze dryer to finally obtain a sustained-release microsphere freeze-dried powder.

### Comparative Example 2

A preparation method for an octreotide acetate sustained-release microsphere is provided. A specific embodiment is the same as Comparative Example 1, but has the difference that the external aqueous phase was an acetic acid buffer solution (pH value: 4.5) containing 3 wt % of a poloxamer surfactant and 10 wt % of sodium chloride.

### Performance Tests

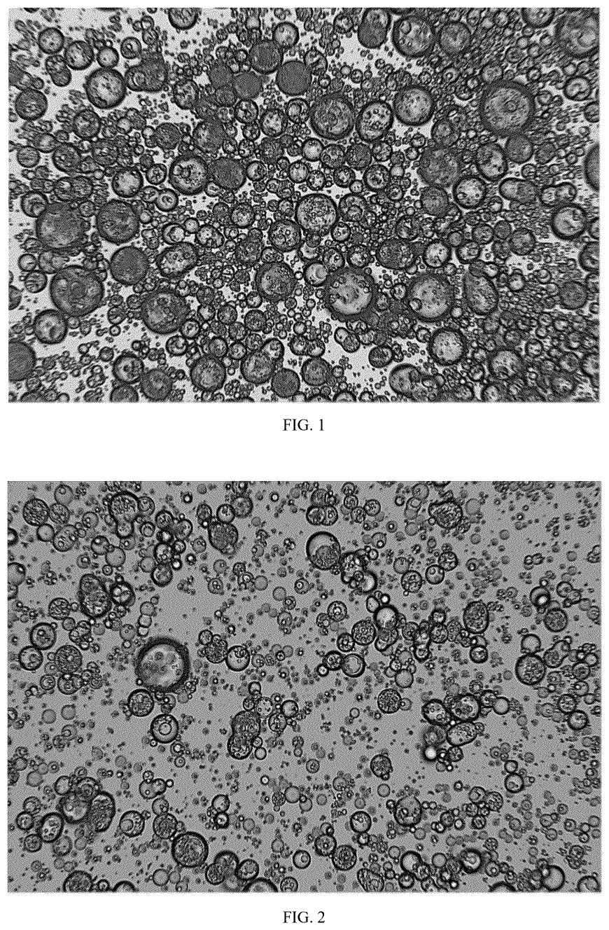

The following tests were carried out on the octreotide acetate sustained-release microspheres prepared in examples and comparative examples.
(1) Measurement of morphology: A WD300-48LT biological microscope was used for measurement, see FIGS. 1-2.
(2) Measurement of particle size: 50 mg of octreotide acetate sustained-release microspheres were each added into an aqueous solution of Tween 80 (Tween 80 content: 0.5 wt %), the particle size was measured for 3 times in average by a particle size laser spectrophotometer after each sample was uniformly dispersed, and a mean value was selected as a measurement result.
(3) Measurement of drug loading rate and dissolution rate: Measurement was performed by liquid chromatography with C18 column at a detection wavelength of 210 nm.
Mobile phase A: tetramethylammonium hydroxide solution (20 mL of a 10% tetramethylammonium hydroxide solution was taken and added into 880 mL of water, and the pH value was adjusted to 5.4 with a 10% phosphoric acid solution)-acetonitrile (900:100) (V/V).
Mobile phase B: tetramethylammonium hydroxide solution (20 mL of a 10% tetramethylammonium hydroxide solution was taken and added into 380 mL of water, and the pH value was adjusted to 5.4 with a 10% phosphoric acid solution)-acetonitrile (400:600) (V/V).

### Gradient Elution:

| Time (min) | Mobile phase A (wt %) | Mobile phase B (wt %) |
|---|---|---|
| 0 | 73 | 27 |
| 30 | 55 | 45 |
| 31 | 73 | 27 |
| 37 | 73 | 27 |

Flow rate: 0.2 mL/min, and column temperature: 30° C.

$$Drug loading rate = ( drug content in microsphere / total weight of microsphere ) \times 100 \% .$$

$$Encapsulation rate = ( actual drug loading rate / theoretical drug loading rate ) \times 100 \% .$$

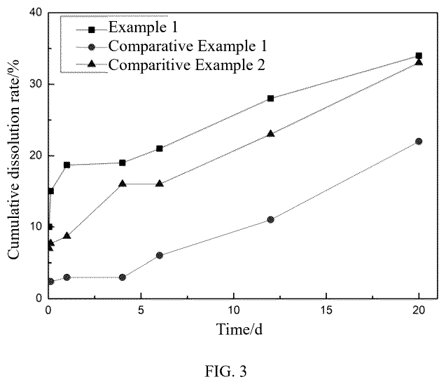

Cumulative dissolution rate after each time point (%)=100%×(C1+C2+ . . . Cn)×V/L, wherein Cn is a drug concentration after removal at each time point, V is a fixed sampling volume at each time point (because the sample is completely removed and then supplemented with the same volume of a medium, the sampling volume is equivalent to the volume of a dissolved medium), and L is a total content of a drug input. Experimental results are shown in FIG. 3.

| | Drug loading | Encapsulation | Particle | |
|---|---|---|---|---|
| Example | rate (%) | rate (%) | size (μm) | Morphology |
| Example 1 | 7.59 | 93 | 43 | Spherical |
| Example 2 | 8.4 | 78 | 27 | Spherical |
| Example 3 | 4.8 | 84 | 59 | Spherical |
| Example 4 | 7.78 | 72 | / | Spherical |
| Example 5 | 7.56 | 70 | / | Spherical |
| Example 6 | 6.88 | 86 | / | Spherical |
| Comparative | 1.5 | 20 | 38 | Non- |
| Example 1 | | | | Spherical |
| Comparative | 4.23 | 68 | 35 | Spherical |
| Example 2 | | | | |

In Example 1 to Example 3, an experiment of changing the concentration of the polylactic acid-co-glycolic acid copolymer (PLGA) finds that when the concentration of PLGA is higher, the particle size of the microsphere prepared is larger, because the increase of the concentration of PLGA leads to the increase of the viscosity of the oil phase 1, the viscosity of PLGA is larger, and the particle size of the microsphere prepared is larger. When the concentration of PLGA is relatively small, the drug is likely to escape into the aqueous phase, resulting in a lower drug loading rate. When the concentration of PLGA is 43%, the average particle size of the microsphere prepared is 43 μm.

In Example 2, Example 4 and Example 5, by changing the pH values of the buffer solutions in the aqueous phase and the curing phase, the drug loading rate and encapsulation rate of the microsphere prepared are decreased when the pH value is 3.0 or 6.0. The reason may be that due to poor stability of octreotide in the aqueous phase and the curing phase under strong acid or weak acid conditions, the octreotide is likely to escape, and as a result, the drug loading rate and encapsulation rate of the microsphere prepared are relatively low. When the pH values of the aqueous phase and the external aqueous phase are 4.5, the drug loading rate and encapsulation rate of the microsphere prepared are highest, and the encapsulation rate reaches 93%.

The drug loading rate and encapsulation rate of the microspheres prepared by a single emulsion method in comparative examples are relatively low, and the reason may be that the octreotide is a hydrophilic drug and is extremely unstable. By using a double emulsification method and adding certain proportions of the surfactant and the metal salt into the external aqueous phase and the curing phase, the stability of the octreotide during emulsification and curing is protected, and the octreotide is prevented from being affected by a water-oil interface and a stirring shear force, thereby improving the drug loading rate and encapsulation rate of the octreotide.

Cumulative dissolution rate/%

| | | | |
|---|---|---|---|
| Time | | Comparative | Comparative |
| point | Example 1 | Example 1 | Example 2 |

| | | | | |
|---|---|---|---|---|
| 1 | h | 10 | 2.4 | 7 |
| 3 | h | 15 | 2.4 | 7.7 |
| 1 | d | 18.7 | 3 | 8.7 |
| 4 | d | 19 | 3 | 16 |
| 6 | d | 21 | 6 | 16 |
| 12 | d | 28 | 11 | 23 |
| 20 | d | 34 | 22 | 33 |

As can be seen from the above analysis, the particle size of the microsphere prepared in the present disclosure is mainly in 30-60 μm, the drug loading rate can reach 7.59%, and the encapsulation rate reaches 93%. The drug microsphere is spherical and has a good encapsulation effect and high repeatability, and drug release in vitro is stable. The present disclosure has the potential for further scale-up.

What is claimed is:

1. A preparation method for an octreotide acetate sustained-release microsphere, wherein the preparation method comprises the following steps:
S1, dissolving a polylactic acid-co-glycolic acid copolymer in a first organic solvent to prepare an oil phase 1; and dissolving octreotide acetate in a second organic solvent to prepare an oil phase 2;
S2, adding the oil phase 2 into the oil phase 1 under stirring conditions to obtain a suspension containing octreotide acetate;
S3, transferring the suspension to an aqueous phase, and performing emulsifying to obtain a semi-cured drug-loaded microsphere; and
S4, transferring the semi-cured drug-loaded microsphere to a curing phase, and removing the solvents to obtain the octreotide acetate sustained-release microsphere.

2. The preparation method of claim 1, wherein a relative molecular mass of the polylactic acid-co-glycolic acid copolymer is 12,000-36,000, a molar ratio of lactic acid to glycolic acid is (50-75):(25-50), and an intrinsic viscosity is 0.15-0.45 dL/g.

3. The preparation method of claim 2, wherein the first organic solvent comprises at least one of ethyl acetate, ethyl formate, methyl formate, methyl acetate, butanone, tetrahydrofuran, acetone, acetonitrile, dimethyl sulfoxide, dichloromethane, chloroform, trichloroethylene, ethylene glycol ether, and triethanolamine.

4. The preparation method of claim 2, wherein the second organic solvent comprises at least one of dimethyl sulfoxide, N,N-dimethylformamide, and N-methylpyrrolidone.

5. The preparation method of claim 4, wherein both the aqueous phase and the curing phase are a buffer solution containing a surfactant and a metal salt.

6. The preparation method of claim 5, wherein a mass percentage of the polylactic acid-co-glycolic acid copolymer in the oil phase 1 is 30%-60%.

7. The preparation method of claim 5, wherein the surfactant comprises at least one of Tween, Span, PVA, poloxamer, and betaine.

8. An octreotide acetate sustained-release microsphere produced by the preparation method of claim 1, wherein an encapsulation rate of the sustained-release microsphere is greater than 90%.

9. A drug, comprising the octreotide acetate sustained-release microsphere of claim 8 and a pharmaceutically acceptable excipient.
